(19) United States
(12) Patent Application Publication
(10) Pub. No.: US 2006/0205066 A1
(43) Pub. Date: Sep. 14, 2006

(54) One touch-type transport medium vessel

(76) Inventor: Han Rim Yum, Seoul (KR)

Correspondence Address: LADAS & PARRY LLP, 224 SOUTH MICHIGAN AVENUE, SUITE 1600, CHICAGO, IL, 60604, US

(21) Appl. No.: 10/550,431

(22) PCT Filed: Dec. 10, 2004
(86) PCT No.: PCT/KR04/03259

§ 371 Date: Sep. 21, 2005

(30) Foreign Application Priority Data
Dec. 31, 2003 (KR) 20-2003-0040924

(51) Int. Cl. C12M 1/26; C12M 1/24

(52) U.S. Cl. 435/309.1; 435/304.2

(57) ABSTRACT

The present invention relates to an one touch-type specimen transport culture medium vessel, including a rod assembly member including a pole type rod having a sterilized collection unit and a rod head; a grip unit; a grip cap; a transparent tube and a culture medium, wherein an adapter is selectively mounted within a transparent tube. More particularly, in a collection preparation step, a sterilized rod and a collection unit are kept in a culture medium with them being not locked in a transparent tube. After the tube returns to its original position after collection, the collection unit of the rod is locked in the culture medium by means of the one-touch operation. In a specimen transport culture medium vessel according to the present invention, a rod is provided with it being contained in a tube. Thus, a plurality of transport culture medium vessels can be packaged on a 10 or 20 unit basis or 100 unit basis. It is thus possible to solve inconvenience that envelopes have to be opened one by one. Upon collection, the rod is collected from the tube separately. After collection, the rod is recombined with the tube at its original place, thus removing unnecessary operation to replace the rod assembly member and the tube. Therefore, secondary infection or contact can be prevented.

### ONE TOUCH-TYPE TRANSPORT MEDIUM VESSEL

1. Technical Field

The present invention relates to a vessel containing a culture medium, for collecting and transporting the culture medium.

2. Background Art

In all culture mediums for microorganism test, in order to obtain accurate results, it is necessary to send collected microorganism to a test room for immediate test. When immediate test is impossible, however, collected microorganism has to be preserved or transported in an adequate environment.

In order to transport the culture medium, a variety of devices such as a syringe and a sterilized plastic bottle are used depending upon the type (blood, pus, urine, stool, etc.) of a culture medium and culture environment (anaerobic, aerotropic, virus, etc.) of transported bacillus. However, a commercialized transport culture medium vessel including a plastic tube for containing various bacillus culture medium and a disposable sterilized rod is used a lot.

In a conventional transport culture medium vessel (EP Publication No. 0643131 A219950315EP), a plastic tube where a rod coupled to a cork and a culture medium are mounted within an envelope in an one package unit is separated additionally and provided as one set in a collection preparation step, and in a culture medium collection preparation step, the envelope is opened, the culture medium is collected using the rod, inserted into the plastic tube having the culture medium mounted in, and then transported by closing the cork coupled to the rod.

Accordingly, in the conventional specimen transport culture medium vessel, the rod is provided as one set with them separated from the tube. Thus, the rod has to be packaged individually so as to prevent contamination of bacillus. If the rod is left alone for a long time with the envelope opened, there is a possibility that the rod is contaminated with other bacillus. Thus, resources occurs since a transport culture medium vessel, which is left along for a long time with the envelope opened, has to be abandoned. Further, when collecting a specimen, an envelope must be opened with sanitary gloves being worn. If the number of collections is a lot, it is very inconvenient for a user to open the sealed envelopes one by one.

Moreover, in the conventional transport culture medium vessel, when the rod in which specimens is collected is inserted into the tube, the culture medium is severely shaken by the collection unit of the rod. The specimens are lost in the culture medium, or are diluted in the culture medium. Accordingly, there is a problem in that it is difficult to preserve the specimens perfectly.

[Disclosure]

[Technical Problem]

Accordingly, the present invention has been made to solve the problems that sealed envelopes must be opened one by one when the number of specimens is a lot and the specimens are difficult to preserve perfectly since the specimens are lost in a culture medium or are diluted in the culture medium, in the conventional transport culture medium vessel. It is an object of the present invention to provide an one touch-type specimen transport culture medium vessel in which a rod is provided with it being kept in a tube, and the rod is collected with it being separated from the tube.

Another object of the present invention is to provide an one touch-type specimen transport culture medium vessel in which after collection, a rod is recombined with a tube, and the top of the rod is pushed by one touch, whereby a collection unit of the rod is locked in the culture medium.

[Technical Solution]

To achieve the above objects, according to the present invention, there is provided an one touch-type specimen transport culture medium vessel, including a rod assembly member including a pole type rod having a sterilized collection unit and a rod head; a grip unit; a grip cap; a transparent tube and a culture medium, wherein an adapter is selectively mounted within a transparent tube.

More particularly, the present invention relates to the structure of the culture medium transport culture medium vessel. In a supply state, i.e., in a collection preparation step, a sterilized rod and a collection unit are kept in a culture medium with them being not locked in a transparent tube. After the tube returns to its original position after collection, the collection unit of the rod is locked in the culture medium by means of the one-touch operation.

### DESCRIPTION OF DRAWINGS

Further objects and advantages of the invention can be more fully understood from the following detailed description taken in conjunction with the accompanying drawings in which:

FIG. 1 shows a state where a rod head and a grip cap are assembled into a grip unit according to a first embodiment of the present invention;

FIG. 2 is a cross-sectional view showing a state before an one touch-type specimen transport culture medium vessel is collected (before use) according to a first embodiment of the present invention;

FIG. 3 is a cross-sectional view showing a state after the one touch-type specimen transport culture medium vessel is collected (after use) according to a first embodiment of the present invention;

FIG. 4 shows a state where the transparent tube of the one touch-type specimen transport culture medium vessel is separated according to a first embodiment of the present invention;

FIG. 5 shows a state where a bacterial strain is collected in a collection unit of the one touch-type specimen transport culture medium vessel according to a first embodiment of the present invention;

FIG. 6 is a view showing a state where the collection unit is contained in a culture medium by depressing the rod head of the one touch-type specimen transport culture medium vessel according to a first embodiment of the present invention;

FIG. 7 shows the shape of the adapter within the transparent tube of the one touch-type specimen transport culture medium vessel according to a second embodiment of the present invention; and

FIG. 8 is a view showing a state where the collection unit of the one touch-type specimen transport culture medium vessel is contained in the liquid culture medium through the adapter according to a second embodiment of the present invention.

### MODE FOR INVENTION

The present invention will now be described in detail in connection with preferred embodiments with reference to the accompanying drawings.

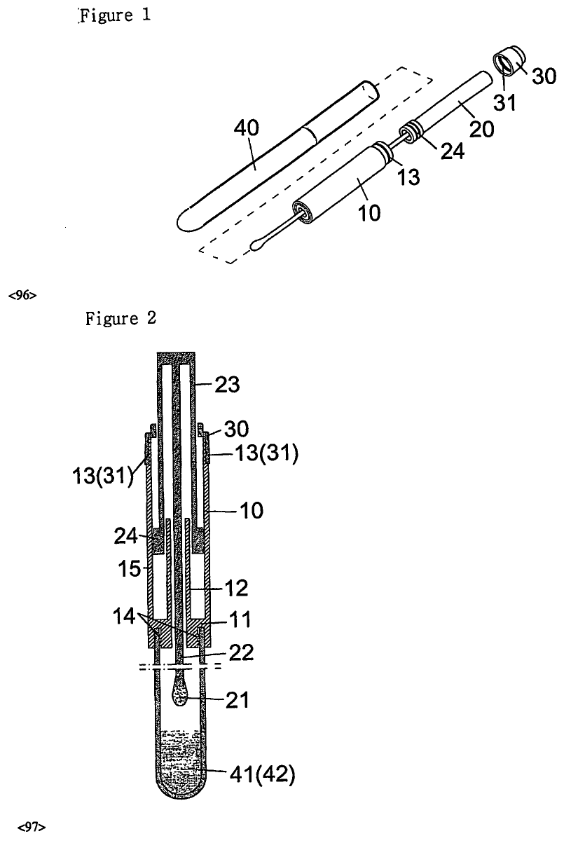

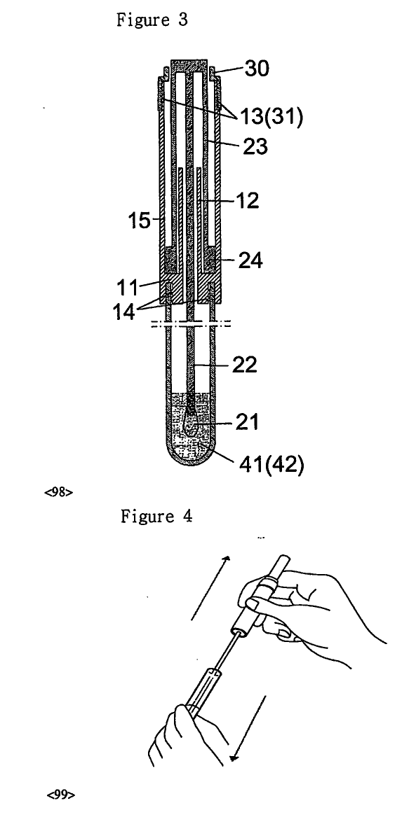

FIG. 1 shows a state where a rod assembly member 20 and a grip cap 30 are assembled into a grip unit 10 according to a first embodiment of the present invention. FIG. 2 is a cross-sectional view showing a state before an one touch-type specimen transport culture medium vessel is collected (before use) according to a first embodiment of the present invention. FIG. 3 is a cross-sectional view showing a state after the one touch-type specimen transport culture medium vessel is collected (after use) according to a first embodiment of the present invention.

The rod assembly member 20 includes a sterilized collection unit 21, a rod 22 and a rod head 23.

The rod head 23 has an up and down long cylindrical shape. A jaw 24 is installed on a lower cylindrical circumference of the rod head 23. Then, when the rod head descends, the jaw 24 is closely adhered to an inner wall of a grip unit. In order to assist the flow of air occurring at this time, the jaw 24 preferably has two or more grooves up and down. The rod 22 including the sterilized collection unit 21 is integrally form at an inner ceil of the rod head 20. In order to prevent leakage of the culture medium, when the rod head 23 descends, the rod 22 is coupled to a cylindrical bobbin 12 from a grip unit base 11. After the rod head 23 descends, the rod 22 is stopped at the grip unit base 11.

A grip cap 30 has a cap shape having its central portion punctured. A groove 31 is disposed at the bottom of the grip cap 30 so that it is coupled to a circular protrusion 13 at the top of the grip unit 10.

When the rod assembly member 20 is coupled to the grip unit 10, the groove 31 that concavely internally touches the grip cap 30 and the protrusion 13 disposed at the top of the grip unit 10 are combined with the grip cap 30 being inserted into the top of the rod head 23. Thus, the rod assembly member 20 is coupled to the grip unit 10.

The grip unit 10 is made of a cylindrical synthetic resin material, and surrounds and protects the rod assembly member 20. The grip unit 10 becomes a downward movement passage of the rod assembly member 20. A circular protrusion 13 is formed on the top of the grip unit 10 and is thus inserted into the groove 31 installed in the grip cap 30, whereby the rod assembly member 20 and the grip unit 10 are coupled.

The bobbin 12 is integrally formed within the grip unit 10 based on the grip unit base 11. When the rod assembly member 20 is downwardly moved, the rod head 23 and the bobbin 12 are coupled.

The bobbin 12, which is integrally disposed in the base 11 within the grip unit 10 together with the grip unit, is disposed from the grip base toward the top in a cylindrical manner. When the rod head 23 descends, the bobbin 12 inserted into the rod head, so that the rod 22 passes through the center of the bobbin 12.

Two or more concave protrusions 14 are formed at the bottom of the grip unit 10, and thus serve to fasten coupling with the transparent tube 40. In order to prevent the leakage of a liquid culture medium, it is preferred that a U-shaped groove is formed in the concave protrusions 14.

The transparent tube 40 is made of a transparent synthetic resin material. The bottom of the transparent tube 40 is filled with a culture medium 41. If the culture medium is a liquid, foamed resin 42 (so-called sponge) is built within the bottom of the transparent tube 40, thus preventing the fluctuation of the liquid culture medium.

A bacterial strain culture medium is made of existing composition. The existing culture medium can have the following types. The present invention employs these compositions.

1. Semi-Fixed Culture Medium.

(1) Carry Blair Type

Sodium thioglycollate 1.5 g, Disodium phosphate 1.1 g, Sodium chloride 5 g, Agar 5 g, Distilled Water 991 ml

The culture medium vessel is fabricated. 1% Calcium chloride of 9 ml is added and pH is set to 8.4. Calcium chloride of 7-9 ml is contained into the screw cap tube and left in hot water for 15 minutes. It is kept at room temperature.

(2) Amies Transport Medium (without Charcoal) Type

Sodium chloride 3.0 g, Sodium hydrogen phosphate 1.15 g, Potassium dihydrogen phosphate 0.2 g, Potassium chloride 0.2 g, Sodium thioglycollate 1.og, Cacium chloride 0.1 g, Magnesium chloride 0.1 g, Agar 4.0 g, Final pH 7.2±0.2 at 25° C.

The reagent component is mixed with distilled water of 1 L, and is then boiled until the reagent component is completely melted.

After being completely melt, the reagent component of 5-6 ml is distributed in the plastic vessel or the glass vessel, and is then covered with a cork. It is then sterilized at high pressure at a temperature of 121° C. at an atmosphere of 151 b, and is then cooled for 15-20 minutes.

(3) Amies Transport Medium (with Charcoal) Type

Charcoal pharmaceutical 10.0 g, Sodium chloride 3.0 g, Sodium hydrogen phosphate 1.15 g, Potassium dihydrogen phosphate 0.2 g, Potassium chloride 0.2 g

Sodium thioglycollate 1.0 g, Cacium chloride 0.1 g

Magnesium chloride 0.1 g, Agar 4.0 g, Final pH 7.2±0.2 at 25° C.

The reagent component is contained in distilled water of 1 L is then mixed. It is boiled until the reagent component is completely melted. After being completely melt, the reagent component of 5-6 ml is distributed in the plastic vessel or the glass vessel, and is then covered with a cork. It is then sterilized at high pressure at a temperature of 121° C. at an atmosphere of 151 b, and is then cooled for 15-20 minutes.

(4) Stuart's Transport Medium Type

Sodium thioglycollate 3.0 g, Sodium glycerophosphate 1.0 g, Cacium chloride 10.0 g, Methylene blue 0.1 g, Agar 3.0 g, Final pH 7.3±0.2 at 25° C.

The reagent component is contained in distilled water of 1 L is then mixed. It is boiled until the reagent component is completely melted. After being completely melt, the reagent component of 5-6 ml is distributed in the plastic vessel or the glass vessel, and is then covered with a cork. It is then sterilized at high pressure at a temperature of 121° C. at an atmosphere of 151 b, and is then cooled for 15-20 minutes.

2. Liquid Culture Medium

(1) Carry Blair Type

Sodium thioglycollate 1.5 g, Disodium phosphate 1.1 g, Sodium chloride 5 g

Distilled Water 991 ml

The culture medium vessel is fabricated. 1% Calcium chloride of 9 ml is added and pH is set to 8.4. Calcium chloride of 7-9 ml is contained in the screw cap tube and left in hot water for 15 minutes. It is kept at room temperature.

(2) Amies Transport Medium (without Charcoal) Type

Sodium chloride 3.0 g, Sodium hydrogen phosphate 1.15 g, Potassium dihydrogen phosphate 0.2 g, Potassium chloride 0.2 g, Sodium thioglycollate 1.og, Cacium chloride 0.1 g, Magnesium chloride 0.1 g, Final pH 7.2±0.2 at 25° C.

The reagent component is mixed with distilled water of 1 L, and is then boiled until the reagent component is completely melted.

After being completely melt, the reagent component of 5-6 ml is distributed in the plastic vessel or the glass vessel, and is then covered with a cork. It is then sterilized at high pressure at a temperature of 121° C. at an atmosphere of 151 b, and is then cooled for 15-20 minutes.

(3) Amies Transport Medium (with Charcoal) Type

Charcoal pharmaceutical 10.0 g, Sodium chloride 3.0 g, Sodium hydrogen phosphate 1.15 g, Potassium dihydrogen phosphate 0.2 g, Potassium chloride 0.2 g, Sodium thioglycollate 1.0 g, Cacium chloride 0.1 g, Magnesium chloride 0.1 g, Final pH 7.2±0.2 at 25° C.

The reagent component is contained in distilled water of 1 L is then mixed. It is boiled until the reagent component is completely melted. After being completely melt, the reagent component of 5-6 ml is distributed in the plastic vessel or the glass vessel, and is then covered with a cork. It is then sterilized at high pressure at a temperature of 121° C. at an atmosphere of 151 b, and is then cooled for 15-20 minutes.

(4) Stuart's Transport Medium Type

Sodium thioglycollate 3.0 g, Sodium glycerophosphate 1.0 g, Cacium chloride 10.0 g, Methylene blue 0.1 g, Final pH 7.3±0.2 at 25° C.

The reagent component is contained in distilled water of 1 L is then mixed. It is boiled until the reagent component is completely melted. After being completely melt, the reagent component of 5-6 ml is distributed in the plastic vessel or the glass vessel, and is then covered with a cork. It is then sterilized at high pressure at a temperature of 121° C. at an atmosphere of 151 b, and is then cooled for 15-20 minutes.

Other virus transport media have been developed.

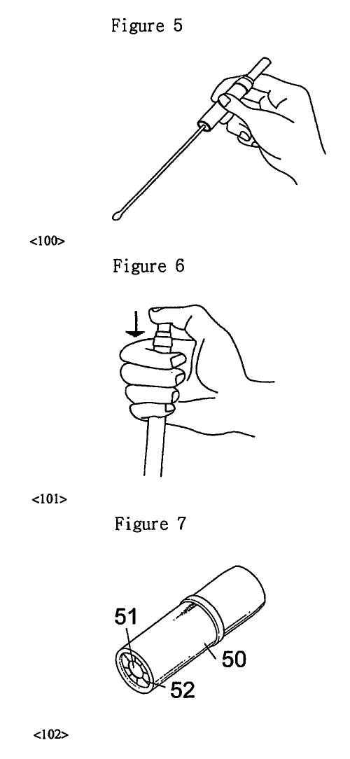

The collection unit 21 in the transparent tube 40 of a not-used transport culture medium vessel, which is provided as a product, is assembled with it spaced from the culture medium 41 in the transparent tube (see FIG. 2). When the culture medium is collected, the transparent tube 40 is separated (FIG. 4) and bacterial strain is thus collected from the collection unit 21 (see FIG. 5). The transparent tube 40 is then recombined to its original place. After the transparent tube 40 is recombined to its original place, if a user downwardly manipulates the rod head 23 in one touch, the rod assembly member 20 is downwardly moved, so that the collection unit 21 is lockted in the culture medium (see FIG. 6). At this time, the downwardly moved rod head 23 is stopped at the grip unit base 11, so that the rod assembly member 20 does not return to its original place due to friction force between the grip unit 10 wall and the jaw 24 at the bottom of the rod head 23.

A second embodiment of the present invention will now be described in connection with the first embodiment of the present invention with reference to the drawings.

The second embodiment of the present invention is different from the first embodiment of the present invention in that the adapter 50 is disposed within the transparent tube 40 in order to prevent the liquid culture medium within the tube from flowing backward over the adapter.

In case of the second embodiment of the present invention, the culture medium 41 can be used only with the liquid culture medium without an anti-flow agent (solid culture medium or foamed resin).

FIG. 7 shows the shape of the adapter 50 disposed within the transparent tube 40.

The adapter 50 has a circular small hole 50 formed at its center. Radial cut lines 52 are formed around the hole 51 so that the surface tension of the liquid culture medium is formed. This device serves to prevent the liquid culture medium 41 at the bottom of the transparent tube forming leaking toward the top of the adapter 50 by using the surface tension of the liquid culture medium contained in the adapter 50.

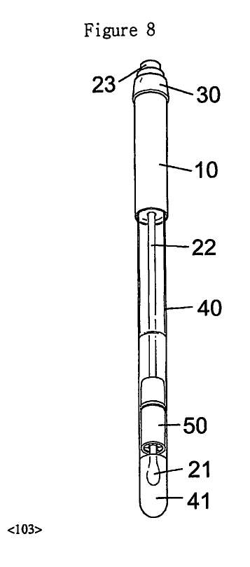

FIG. 8 is a view showing a state where the collection unit 21 is contained in the liquid culture medium 41 through the adapter 50.

The collection unit 21 in the transparent tube 40 of a not-used transport culture medium vessel, which is provided as a product, is assembled with it spaced from the culture medium 41 in the transparent tube (see FIG. 2). When the culture medium is collected, the transparent tube 40 is separated (FIG. 4) and bacterial strain is thus collected from the collection unit 21 (see FIG. 5). The transparent tube 40 is then recombined to its original place. After the transparent tube 40 is recombined to its original place, if a user downwardly manipulates the rod head 23 in one touch, the rod assembly member 20 is downwardly moved, so that the collection unit 21 is lockted in the culture medium (see FIG. 6). At this time, the downwardly moved rod head 23 is stopped at the grip unit base 11, so that the rod assembly member 20 does not return to its original place due to friction force between the grip unit 10 wall and the jaw 24 at the bottom of the rod head 23.

### INDUSTRIAL APPLICABILITY

In a specimen transport culture medium vessel according to the present invention, a rod is provided with it being contained in a tube. Thus, a plurality of transport culture medium vessels can be packaged on a 10 or 20 unit basis or 100 unit basis. It is thus possible to solve inconvenience that envelopes have to be opened one by one.

Upon collection, the rod is collected from the tube separately. After collection, the rod is recombined with the tube at its original place, thus removing unnecessary operation to replace the rod assembly member and the tube. Therefore, secondary infection or contact can be prevented.

After collection, the operation to contain the collection unit of the rod in the culture medium is performed by pushing the head of the rod assembly member in one touch. The fluctuation of the culture medium can be minimized and loss of the culture medium can be thus minimized.

1. An one touch-type specimen transport culture medium vessel including a rod assembly member, a transparent tube and a culture medium, comprising:
a rod assembly member including a pole type rod having a sterilized collection unit and a rod head;
a grip unit;
a grip cap;
a transparent tube; and
a culture medium,
wherein if the rod head is pressed, a collection unit is contained in the culture medium within the transparent tube while the rod assembly member downwardly moves within the grip unit.

2. The one touch-type specimen transport culture medium vessel as claimed in claim 1, wherein the culture medium within the transparent tube is filled with a semi-solid material.

3. The one touch-type specimen transport culture medium vessel as claimed in claim 1, wherein the culture medium within the transparent tube is a liquid phase and is filled with foamed resin.

4. The one touch-type specimen transport culture medium vessel as claimed in claim 1, further comprising an adaptor disposed within the transparent tube, for preventing an upstream current of the culture medium.

5. The one touch-type specimen transport culture medium vessel as claimed in claim 4, wherein the culture medium within the transparent tube is a liquid phase and is filled with foamed resin.

6. The one touch-type specimen transport culture medium vessel as claimed in claim 4, wherein the culture medium within the transparent tube is filled with a liquid material.
